(19) United States
(12) Patent Application Publication
(10) Pub. No.: US 2006/0147550 A1
(43) Pub. Date: Jul. 6, 2006

(54) Immune system enhancement and stimulation formulation

(76) Inventor: Wayne F. Gorsek, Boynton Beach, FL (US)

Correspondence Address: HOFFMAN WASSON & GITLER, P.C;CRYSTAL CENTER 2, SUITE 522, 2461 SOUTH CLARK STREET, ARLINGTON, VA, 22202-3843, US

(21) Appl. No.: 11/024,495

(22) Filed: Dec. 30, 2004

(51) Int. Cl. A61K 33/04; A61K 33/32; A61K 31/715; A61K 31/355; A61K 31/343; A61K 31/12

(52) U.S. Cl. 424/641; 424/702; 514/54; 514/474; 514/458; 514/690

(57) ABSTRACT

The composition of ingredients enhances and stimulates the immune system. The composition includes beta glucan and coenzyme Q10 (Ubiquinone). Both of these components have the effect of preventing, reversing and treating tumors. In addition, secondary nutrients, including Vitamin C, Vitamin E, Selenium and Zinc, offer additional benefits when combined with beta glucan and coenzyme Q10.

### BACKGROUND OF THE INVENTION

The invention relates to a composition for enhancing and stimulating the immune system.

The immune system protects the body from damage caused by bacteria, viruses, parasites, environmental toxins and other foreign bodies to which the body is exposed daily. Optimum health requires a properly functioning immune system. In the absence of a properly functioning immune system, a person is subjected to repeated and serious illnesses, which, alone, or having a cumulative effect, reduce the overall health of the person.

The immune system is a complex network of soluble factors, cells and tissues recognizing and responding to a wide range of potential harmful foreign agents. When exposed to a foreign body, the immune system responds with a two-step response. Initially, a non-specific inflammatory response isolates the virus and destroys it with the assistance of antigen specific immune cells. Secondly, the immune system develops a resistance to the antigen to which it has been exposed by producing and circulating antibodies. These antibodies will respond quicker and more efficiently when exposed to the same antigen.

Inherently, some people have immune systems functioning better and more efficiently than other persons. Factors determining the performance of the immune system include heredity, prior diseases and illnesses, nutrition and environmental factors, such as the number of antigens exposed to the person. It is possible to ingest substances to allow the immune system to operate at peak performance.

It is an object of the present invention to provide a formulation for enhancing a person's immune system.

It is another object of the invention to provide a formulation having beta glucan, which is shown to have anti-tumor and anti-microbial properties.

It is another object of the invention to provide a formulation of vitamins and nutrients having a synergistic result among its various components.

These and other objects of the invention will become apparent to one of ordinary skill in the art after reading the disclosure of the invention.

### SUMMARY OF THE INVENTION

The formulation which includes an unique combination of ingredients enhances and stimulates the immune system. The formulation includes beta glucan and coenzyme Q10 (Ubiquinone). Both of these components combine to have the effect of preventing, reversing and treating tumors. In addition, secondary nutrients, including Vitamin C, Vitamin E, Selenium and Zinc, offer additional added benefits when combined with beta glucan and coenzyme Q10.

### DETAILED DESCRIPTION OF THE INVENTION

Beta glucan has an active ingredient of long chain 1,3 linkage and 1,6 side branching linkage. Beta glucan exhibits effective immuno stimulating properties. Over 500 publications have reported beta glucan's anti-tumor and anti-microbial activities. Often, beta glucan is used in conjunction with traditional radiation and chemotherapy treatments for cancer.

Beta 1,3-glucans are derived from various bacterial, mushroom, yeast and cereal sources. Highly purified beta glucan can be derived from the cell walls of saccharomyces cerevisiae. Studies have shown that beta glucan from this source to be a potent macrophage stimulant and malignant tumor and growth inhibitor. In addition, beta glucan increases production of three important cytokines (IL-2, TNF-α and IFN-γ). These cytokines play an important role in physiological processes and bioregulation of host defense reactions.

Ubiquinone molecules are also known as coenzyme Q or mitoquinones. They are involved in electron transport in mitochondrial preparations playing an essential role in the oxidation of succinate or NADH via the cytochrome system. They serve not only as a coenzyme but also, in their reduced forms, as antioxidants. They are synthesized de novo in all animal tissues and cannot thus be regarded as vitamins. Ubiquinones are present in all aerobic organisms, bacteria, plants and animals (the name ubiquinone was proposed with reference to thier ubiquitous occurrence).

In order to secure the desired result the following essential components are provided:

Beta Glucan in the amount of 10-10,000 mg per serving/day. Optimally, beta glucan is present in the amount of 500 mg.

Coenzyme Q10 (Ubiquinone) in the amount of 1-10,000 mg per serving/day. Optimally, Coenzyme Q10 is present in the amount of 400 mg. Coenzyme Q10 prevents, reverses, cures and treats cancer of all types.

In addition, the composition has secondary nutrients for providing additional benefits. These include:

Vitamin C: 10-10,000 mg per serving/day; optimally 400 mg per day.

Vitamin E: 10-10,000 IU per serving/day; optimally 100 IU per day.

Selenium: 10 mcg-10 mg per serving/day; optimally 400 mcg.

Zinc: 1 mg-1,000 mg per serving/day; optimally 30 mg.

The unique formulation is administered to a mammalian host orally, preferably in capsule form). A preferred formulation includes the following unique combination of components.

Beta Glucan: 500 mg

Ubiquinone: 400 mg

Vitamin C: 500 mg

Vitamin E: 100 IU

Selenium: 400 mcg

Zinc: 30 mg

1. (canceled)

2. An effective dosage of an immune system enhancement and stimulation formulation, comprising
500 mg beta glucan;
400 mg ubiguinone;
500 mg Vitamin C;
100 IU Vitamin E;
400 mcg Selenium; and
30 mg Zinc.

3. A method for enhancing and stimulating the immune system, comprising orally administering to a mammalian host an effective dosage of a formulation, consisting essentially of
10-10,000 mg beta glucan;
1-10,000 mg Ubiquinone;
10-10,000 mg Vitamin C;
10-10,000 IU Vitamin E;
10 mcg-10 mg Selenium; and
1 mg-1,000 mg Zinc.
